(19) United States
(12) Patent Application Publication
(10) Pub. No.: US 2024/0200267 A1
(43) Pub. Date: Jun. 20, 2024

(54) METHOD AND DEVICE FOR STERILIZING RAW MATERIAL FOR PRODUCING AT LEAST PART OF A CONTAINER COMPRISING FIBERS, METHOD FOR PRODUCING A CONTAINER COMPRISING FIBERS USING THE STERILIZED RAW MATERIAL, AND CONTAINER TREATMENT PLANT

(71) Applicant: Krones AG, Neutraubling (DE)

(72) Inventors: Jörg ZACHARIAS, Kofering (DE); Frank WINZINGER, Regensburg (DE)

(21) Appl. No.: 18/536,004

(22) Filed: Dec. 11, 2023

(30) Foreign Application Priority Data
Dec. 20, 2022 (DE) 10 2022 134 088.0

(51) Int. Cl. D21C 9/08; A61L 2/04; A61L 2/16; A61L 2/26; D21J 3/10

(52) U.S. Cl. CPC D21C 9/083; A61L 2/04; A61L 2/16; A61L 2/26; D21J 3/10

(57) ABSTRACT

The disclosure relates to a method for sterilizing raw material for producing at least a part of a container comprising fibers, wherein the method comprises: providing the raw material and sterilizing the raw material to form sterilized raw material. The disclosure further relates to sterilized raw material for producing at least a part of a container comprising fibers, wherein the sterilized raw material was obtained by means of the method, to a method for producing a container comprising fibers using the sterilized raw material, and to the container comprising fibers. In addition, the disclosure relates to a device for sterilizing raw material for producing at least a part of a container comprising fibers, wherein the device is designed to carry out the method for sterilizing raw material.

### CROSS REFERENCE TO RELATED APPLICATION

The present application claims priority to German Patent Application No. 10 2022 134 088.0 filed on Dec. 20, 2022. The entire contents of the above-listed application are hereby incorporated by reference for all purposes.

### TECHNICAL FIELD

The disclosure relates to a method and to a device for sterilizing raw material for producing at least a part of a container comprising fibers, to a sterilized raw material, to a method for producing a container comprising fibers using the sterilized raw material, to a container comprising fibers, and to a container treatment plant.

### BACKGROUND

For the production of containers comprising fibers, it is known to use cellulose fibers or wood fibers or the like. These fibers can be available together with a fluid as pulp. Depending on the origin of the fibers and, for example, the fluid, a microbiological germ load can exist, for example due to bacteria, molds, yeasts and/or spores. The microbiological germ load can negatively influence a product filled into the container.

### SUMMARY

### Problem

The problem addressed by the disclosure is to provide a method and a device for sterilizing raw material for producing at least a part of a container comprising fibers, a sterilized raw material, a method for producing a container comprising fibers using the sterilized raw material, a container comprising fibers, and a container treatment plant which can reduce microbiological load.

### Solution

The problem is solved by the method and the device for sterilizing raw material for producing at least a part of a container comprising fibers, the sterilized raw material, the method for producing a container comprising fibers using the sterilized raw material, the container comprising fibers, and the container treatment plant as described herein.

The method according to the disclosure for sterilizing raw material for producing at least a part of a container comprising fibers comprises providing the raw material and sterilizing the raw material to form sterilized raw material.

The raw material can thus be sterilized before the production of at least a part of a container comprising fibers. The sterilization of the part of the container comprising fibers or of the entire container after production is then not necessary in certain cases. Alternatively, however, additional sterilization after production can also take place.

The raw material comprises the fibers and can also comprise further substances, such as water or additives. The raw material can be made available as a pulp. The pulp can thus be sterilized before use for the production of a container comprising fibers.

The fibers can comprise lignin, banana leaves and/or quinine. The fibers can comprise, for example, cellulose fibers, fibers from softwoods, broad-leaved woody plants, and/or sycamores, and/or from grasses, reeds, and/or bamboo, or the like. The fibers can comprise silk threads, spider threads, algae, natural fibers (such as silphia fibers, hemp, corn, cotton), banana peels, orange peels, grass, straw, potato starch, or processed cow manure. Also, cellulose fibers can be provided which originate from a process by which they were artificially grown. These alternative materials can completely or partially replace wood as the base material for a fluid mass with fibers when there are material shortages.

The fibers can comprise fiber mixtures made of non-wood material, for example cotton, hemp and/or textile fibers.

For example, the fibers can comprise viscose fibers. A viscose fiber is an artificial fiber made of regenerated cellulose, wherein the viscose fibers have, as a starting base, 100% cellulose treated in a multi-stage method. The viscose fibers can comprise a flat or cable-like structure, a trilobal shape, or a double trilobal structure. The fibers can comprise a flat or hollow structure or a corrugated or rough outer surface.

Various types of fibers can also be used in combination. For example, fibers with different outer surfaces, and/or shapes, and/or a hollow structure, and/or a flat structure, and/or made of different materials can be combined. Different length distributions of the fibers can also be present in this case.

Due to the natural origin of the fibers, they can be biodegradable. In addition, they are sustainable and renewable.

The fibers may be used in a pulp. The pulp can be or comprise a mixture of water, for example with additives, and fibers.

The raw material can comprise bale goods, sack goods, recycled paper, cardboard packagings or also pre-dispersed raw material (in each case in a crushed state), which can carry a microbiological basic load which can contaminate a product filled later into a container comprising the raw material.

The raw material may also be or comprise recycled raw material. The recycled raw material may be sterilized raw material that has already been used, for example residues from a production process of containers comprising fibers and/or containers comprising recycled fibers which comprised sterilized raw material.

Therefore, sterilization of the raw material for producing at least a part of a container comprising fibers is expedient. A heat effect on the container comprising fibers during a drying process after production may not be sufficient with regard to germ killing.

The sterilization can comprise a physical and/or electrothermal sterilization. For example, the physical sterilization can take place by means of a physical sterilizer, such as by means of microwave radiation, by means of an ohmic heater, by means of induction, by means of ultraviolet radiation, by means of infrared light, by means of high-frequency heating, by means of a pulsed electric field, by means of plasma, and/or by means of high pressure.

In this type of sterilization, it is not necessary for additional water to be introduced into the raw material. The physical and/or electrothermal sterilization can be used for raw material of high viscosity.

The physical sterilization can take place on a transfer path of the raw material to the molding machine, for example directly in a supply line system and/or in a unit provided therefor, a mixing container or generally in a tank provided for this purpose.

The sterilization can alternatively or additionally comprise thermal heating. For example, the thermal heating can take place by means of a flash pasteurization device. The raw material may be pumped through and sterilized in the flash pasteurization device before it can enter the mold. Pumps can also be understood to mean conduction by means of overpressure or gravity. The flash pasteurization device can comprise, for example, a tubular heat exchanger (which can protect fibers of the raw material), which can be designed according to the viscosity of the raw material and the heat transfer necessary for the raw material, so that a corresponding reduction in germs can be achieved, or the flash pasteurization device can comprise a plate heat exchanger. The raw material may be brought to at least 72° C. for at least approximately 15 to 30 seconds by the flash pasteurization device. In the case of related applications in beverages, these moderate conditions (temperature of 72°) are also referred to as pasteurization. Therefore, this term is also introduced here in connection with the raw material, since pasteurization can also describe a germ reduction by these and comparable methods. Thereafter, the sterilized raw material can be cooled regeneratively. The flash pasteurization device can use the same heat source as well as the flash pasteurization device provided for the sterilization of the product to be filled. The heat recovery and/or recuperation and/or connection to other heat sources and heat consumers during operation can also be kept in mind. Alternatively, thermal heating may comprise heating the raw material over several hours.

In other words, the sterilization can take place by means of an indirect heat input into the raw material.

For example, the sterilization can take place in a heat exchanger, for example in a tubular heat exchanger, wherein other heat exchanger types may also be possible however, such as a plate heat exchanger or a scraped surface heat exchanger.

For example, a heat retention section can be provided in the form of a line, in which a predetermined temperature of the raw material can be maintained before it can be cooled again. It is not necessary for the raw material to be cooled after the heat retention section. Without cooling, for example the sterility of the raw material can be preserved until it reaches an aseptic room, which is described for example further below in connection with the container treatment plant.

For example, the cooling can take place regeneratively, in that heat from the raw material can be output to a heat exchange medium, which can be used again for heating at another point, for example of new raw material to be sterilized, or in that heat can be output to new raw material to be sterilized. For example, this can take place using what is known as a product-product heat exchanger (portion).

For example, heating of the raw material to at least 75° C., for example to at least 90° C., can take place. Heating of the raw material to at least 110° C. can also take place.

For example, heating of the raw material to the above temperatures can then take place in each case at least for 15 seconds, for example for at least 30 seconds in each case. It may also be the case that longer time durations of more than 60 seconds are provided.

By means of heat loads of different intensities, which can describe the relationship between the temperature profile and the duration of action in beverages, a differently effective germ reduction can be achieved. Therefore, it is noted here that this can be taken into account when selecting the conditions. The dependence on this can be specified by the germs or microbiological load to be expected, which can be different depending on the origin of the raw material and/or the region. As a rule, reference is made to moderate reductions, as are known from aseptic applications of the beverage industry, in orders of magnitude of the reduction between 4 and 6 log stages. It could also be referred to as pasteurization of the raw material. In the case of log stages, reference can be made to sterilization conditions in the order of magnitude of 9-12 log stages, as can be achieved, for example, in the production of sterile water.

Energy used to heat the raw material may be used for further treatment steps when producing a container comprising fibers based on the raw material. For example, for drying and/or heating the mold via a gas-water heat exchanger and/or for heating the molding medium (for example by means of water or air).

The sterilization can alternatively or additionally comprise chemical sterilization. For example, chemical sterilization can take place by means of an addition of chemical disinfectant. For example, the chemical disinfectant can comprise H₂O₂, O₂, O₃, NaClO₂, NaClO, HClO, HOCl-water or electrochemically activated water.

In chemical sterilization, the raw material can be sterilized directly. The chemical disinfectant can be added to a dispersing liquid in a corresponding concentration and act depending on the concentration and exposure time. The chemical sterilization can take place during a mixing process of the raw material.

The raw material may comprise fibers. For example, these may be dry fibers. For example, the fibers can comprise natural fibers. In the case of dry fibers, sterilization can take place, for example, by means of hot air and/or ultraviolet radiation.

The raw material may comprise fibers and a fluid. The mixture of fibers and fluid may constitute a pulp. For example, the fibers can comprise natural fibers. For example, the fluid can comprise water, wherein the fluid may comprise additives for example.

Furthermore, a sterilized raw material for producing at least a part of a container comprising fibers is provided, wherein the sterilized raw material was obtained by means of the method for sterilizing raw material for producing at least a part of a container comprising fibers, as described above or further below.

By means of the sterilized raw material, a cover for a container, for example a container comprising fibers, as described above or further below, can be produced.

Furthermore, a method for producing a container comprising fibers using the sterilized raw material, as described above or further below, is provided. The sterilized raw material can be introduced into a mold and pressed there to form the container comprising fibers.

In addition, a container comprising fibers is provided, wherein at least a part of the container comprising fibers comprises sterilized raw material which was obtained by means of the method as described above or further below.

The container comprising fibers can have been produced by means of the method for producing a container comprising fibers using the sterilized raw material, as described above or further below. The container may comprise a cover.

Furthermore, a device for sterilizing raw material for producing at least a part of a container comprising fibers is provided, wherein the device is designed to carry out the method for sterilizing raw material for producing at least a part of a container comprising fibers, as described above or further below.

The device can further comprise a feed device for raw material and a sterilizing device for sterilizing the raw material. The feed device can comprise a mixing container, for example for receiving the raw material. The raw material may be dispersed in the mixing container.

The sterilizing device can comprise at least one of the following:
a microwave radiation generation device, an ohmic heater, an induction heating device, an ultraviolet radiation generation device, an infrared light generation device, a high frequency generation device, a pulsed electric field generation device, a plasma generation device, a high pressure generation device, a flash pasteurization device, a heat exchanger, a feed device for feeding chemical disinfectant.

If the raw material consists only of dry constituents, sterilization by means of hot gas, for example air, can take place. In this case, the sterilizing device can comprise a blower. A gas burner or a heater or the like can also be part of the sterilizing device.

It may be the case that production of the container can take place both from sterilized and non-sterilized raw material. For example, an outer layer of the container can comprise non-sterilized material and a further inward layer of the container can comprise sterilized material. For example, the inner layer can consist of sterilized material. The inner layer can, for example, come into contact with product filled into the container. The production of the container can take place, for example, in such a way that first non-sterilized material is introduced into a mold and then sterilized raw material in order to form a further inner layer. Between the two steps, an at least partial drying and/or solidification of the outer layer can take place.

It may be the case that the container can be directly subjected to a filling and a closure by a closure means after its molding. Subsequently, a plurality of containers can be packaged together as a batch, and/or stacked on a pallet.

In general, degassing of the raw material is also considered. For example, a degassing container can be arranged between the mixing container and the mold, for example between the mixing container and the heat exchanger, for example also between two sections of the heat exchanger for a preheated raw material, through which degassing container the raw material can be conveyed and in which, for example, a negative pressure can prevail. Alternatively or additionally, the water can be degassed before it comes into the mixing container. It may be the case that the water or the raw material is still heated before degassing, for example to a temperature in the range between 30° C. and 80° C., for example between 50° C. and 70° C. The range limits of the value range can also be included. For example, oxygen can be removed from the material by means of degassing, which oxygen may later be harmful to the beverage after the container has been filled. The degassing container can be connected for example to a vacuum pump. A valve can be present in the inlet of the degassing container. The degassing can also be an independent disclosure.

Furthermore, a container treatment plant is provided which a provision device for providing a sterile pulp comprising fibers, a production device for producing a container comprising the fibers from the sterile pulp comprising fibers, and a filling and closing device for filling the sterile container comprising fibers with product and for closing the filled sterile container comprising fibers, and at least one aseptic room which is designed to maintain a defined aseptic environmental condition for the sterile container comprising fibers, and which extends from the provision device to the filling and closing device.

Filtered air and/or sterile air can be applied to the at least one aseptic room.

The at least one aseptic room can be designed in such a way that the sterility of sterile elements of the container treatment plant, such as machines or materials used, for example, the sterile pulp comprising fibers, can be retained in the aseptic room.

For example, the at least one aseptic room can be a room having a low-contamination environment.

The low-contamination environment, which can be a clean room, for example, can be obtained by one or combinations of the following measures:
The low-contamination environment (for example within a machine protection) can be subjected to an overpressure compared to the ambient atmosphere by, for example, air filtered by fine filters being blown into the aseptic room at least during an operating mode of the container treatment plant (for example production).
Drives for a very wide variety of movements of a mold for producing the container comprising fibers (for example a precoat mold or a mold carrier) and/or transport elements can be arranged outside the aseptic room. For example, the drives can be arranged for opening and closing the mold (cavity) and/or for raising or lowering a container comprising fibers and/or a movement of a filling nozzle outside the aseptic room.
A surface of an inner wall of the aseptic room can be cleaned and/or sterilized at regular intervals. For example, the cleaning and/or the sterilization can take place by spraying or steaming with alkaline solution, acid, disinfectant liquid, hydrogen peroxide (gaseous or liquid), cleaning foam or the like. For example, during the cleaning and/or sterilization, the inner and outer sides of the mold and/or the filling nozzles and/or closing heads can also be included. For example, surfaces of internal walls of the machine protection can also be included.
The low-contamination environment can be sealed against the ambient atmosphere. In the case of a carousel-type machine (a plurality of molds and/or filling nozzles and/or closing heads in each case or together on a carousel), the seal can be designed as a water lock or a rubber seal which can seal the rotating part relative to the stationary part of the carousel-type machine.

The container treatment plant can also comprise a CIP/SIP system (Clean-in-Place/Sterilization-in-Place system) for the molds for producing the containers comprising fibers.

For media such as alkaline solution, water, acid and/or additives, the CIP/SIP system can comprise in each case at least one tank for providing one of the media.

A heat exchanger can be provided in the tank, for example for heating one of the media provided in the tank before it is used.

The CIP/SIP system can comprise a separate circuit for a line system in which the pulp comprising fibers can be transported. For example, the CIP/SIP system can comprise a further separate circuit for a line system in which product can be transported.

It can be provided that the container treatment plant comprises a separate CIP/SIP system for a line system in which the pulp comprising fibers can be transported.

Cleaning/sterilizing of the above-mentioned line systems can take place at a temperature of at least 60° C. or at least 80° C. or at least 120° C.

### BRIEF DESCRIPTION OF THE FIGURES

The accompanying figures show, by way of example, aspects and/or embodiments of the disclosure for better understanding and illustration. In the figures:

FIG. 1 shows a first embodiment of a device for sterilizing raw material for producing at least a part of a container comprising fibers,

FIG. 2 shows a second embodiment of a device for sterilizing raw material for producing at least a part of a container comprising fibers,

FIG. 3 shows a third embodiment of a device for sterilizing raw material for producing at least a part of a container comprising fibers, and

FIG. 4 shows a container treatment plant.

### DETAILED DESCRIPTION OF THE FIGURES

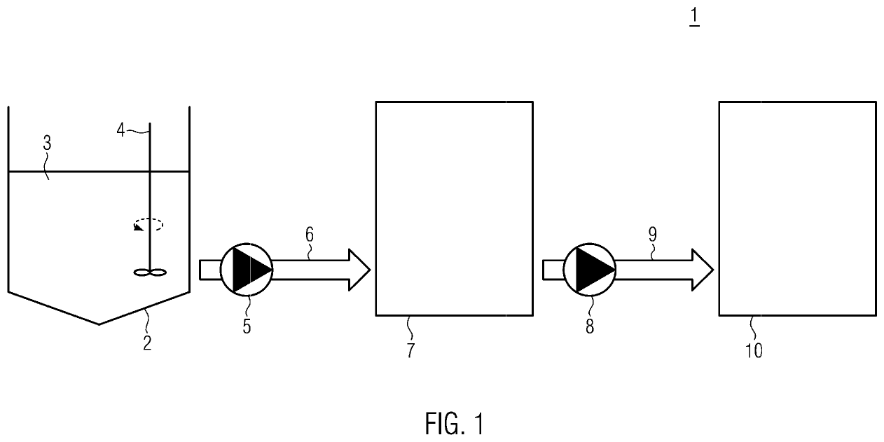

FIG. 1 shows a first embodiment of a device 1 for sterilizing raw material 3 for producing at least a part of a container comprising fibers. The raw material 3 to be sterilized is provided in a mixing container 2, in which it can be dispersed by means of a stirrer 4. Thereafter, the raw material 3 is transported by means of a first pump 5 through a first pipeline 6 to and through a flash pasteurization device 7. The flash pasteurization device 7 may comprise a plate heat exchanger or a tubular heat exchanger. In the flash pasteurization device 7, the raw material is sterilized to form sterilized raw material, for example at a temperature of at least 72° C. for approximately 15 to 30 seconds, and is transported by means of an (optional) second pump 8 through a second pipeline 9 to a production device 10 for producing the at least one part of the container comprising fibers. In the production device 10, the sterilized raw material can be introduced, for example, into a mold, the inner surface of which can correspond at least substantially to a negative form of the at least one part, to be produced, of the container comprising fibers.

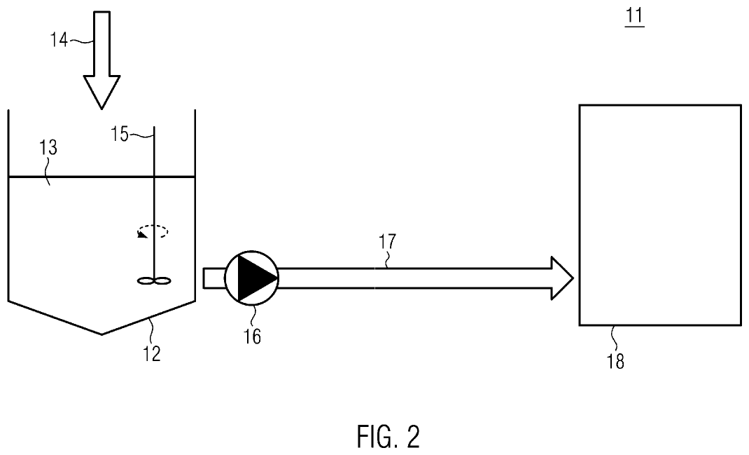

FIG. 2 shows a second embodiment of a device 11 for sterilizing raw material 13 for producing at least a part of a container comprising fibers. The raw material 13 to be sterilized is provided in a mixing container 12, wherein a chemical disinfectant may be added to the raw material 13 in the mixing container 12 by means of a feed device 14 for feeding chemical disinfectant, in order to be able to carry out chemical sterilization. The chemical disinfectant can comprise H₂O₂, O₂, O₃, NaClO₂, NaClO, HClO, HOCl-water or an electrochemically activated water. The mixture of raw material 13 and chemical disinfectant can be dispersed by means of a stirrer 15. During chemical sterilization, the raw material can thus be sterilized directly in the mixing container 12. The chemical disinfectant can be added to a dispersing liquid in a corresponding concentration and act depending on the concentration and exposure time. The sterilized raw material is transported by means of a pump 16 through a pipeline 17 to a production device 18 for producing the at least one part of the container comprising fibers. In the production device 18, the sterilized raw material can be introduced, for example, into a mold, the inner surface of which can correspond at least substantially to a negative form of the at least one part, to be produced, of the container comprising fibers.

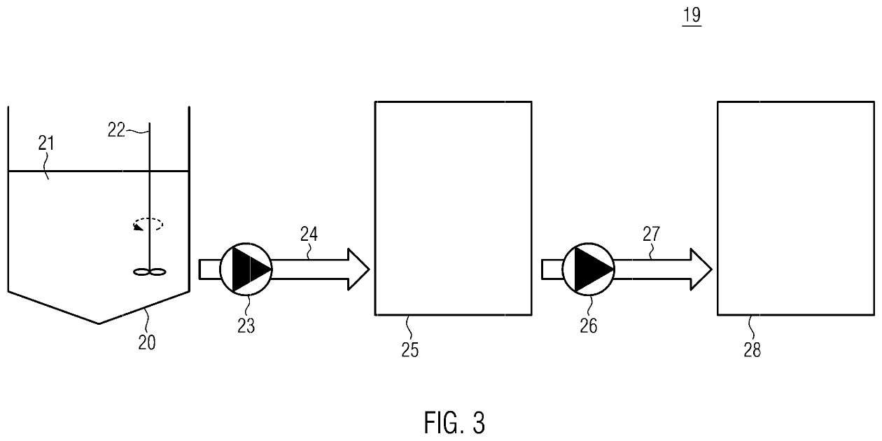

FIG. 3 shows a third embodiment of a device 19 for sterilizing raw material 21 for producing at least a part of a container comprising fibers. The raw material 21 to be sterilized is provided in a mixing container 20, in which it can be dispersed by means of a stirrer 22. Thereafter, the raw material 21 is transported by means of a first pump 23 through a first pipeline 24 to and through a physical sterilizer 25. Here, the physical sterilizer 25 is designed as a unit between the pipelines 24, 27. However, it can also be provided that the physical sterilization can take place in the pipelines, so that no additional unit is required. The physical sterilization can take place by means of microwave radiation, by means of an ohmic heater, by means of ultraviolet radiation, by means of high-frequency heating, by means of a pulsed electric field, by means of plasma and/or by means of high pressure. In the physical sterilizer 25, the raw material 21 is sterilized to form sterilized raw material and is transported by means of an optional second pump 26 through a second pipeline 27 to a production device 28 for producing the at least one part of the container comprising fibers. In the production device 28, the sterilized raw material can be introduced, for example, into a mold, the inner surface of which can correspond at least substantially to a negative form of the at least one part, to be produced, of the container comprising fibers.

Dry raw material can be transported, for example, by means of a blower (not shown).

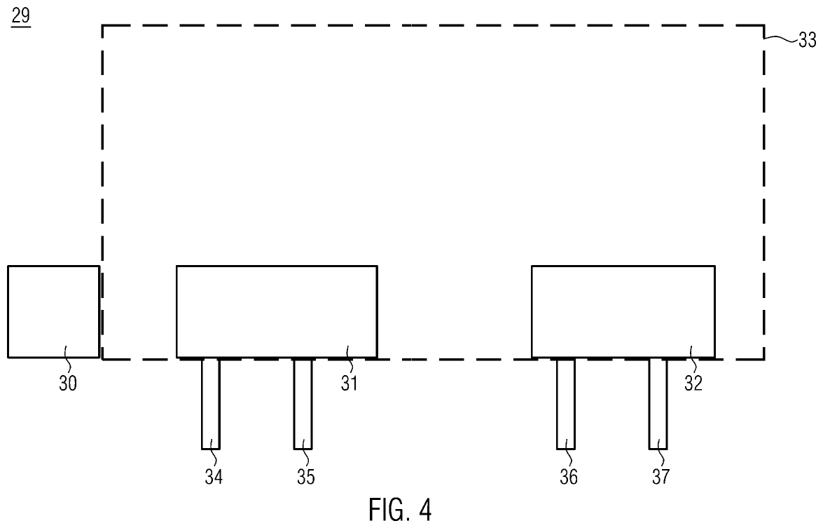

FIG. 4 shows a container treatment plant 29, which comprises a provision device 30 for providing a sterile pulp comprising fibers, a production device 31 for producing a container comprising the fibers from the sterile pulp comprising fibers, and a filling and closing device 32 for filling the sterile container comprising fibers with product and for closing the filled sterile container comprising fibers. From the provision device 30 to the filling and closing device 32, an aseptic room 33 extends, which is designed to maintain a defined aseptic environmental condition for the sterile container comprising fibers.

The sterile pulp comprising fibers can be produced by means of the method steps already described above and then provided, by the provision device 30, for the production of the containers comprising the fibers. In order to be able to ensure the sterility of the pulp comprising fibers and then also of the container comprising fibers produced therefrom, the machines from the provision device 30 up to the filling and closing device 32 are arranged in the aseptic room 33.

For example, the mold (cavity), which can be used to produce a container comprising fibers, can be sterile in order to prevent contamination of the sterile pulp comprising fibers, which can be introduced into the mold during a production process. After production, the container comprising the fibers can be filled with product and then closed, by means of the filling and closing device 32, so that filling and closing also take place under the defined aseptic environmental conditions.

The production device 31 and the filling and closing device 32 are arranged in the aseptic room, wherein drives of the production device 31 and the filling and closing device 32, however, are arranged outside of the aseptic room 33. Apart from the drives, the elements of the production device 31 and the filling and closing device 32 satisfy the defined aseptic environmental condition.

The production device 31 comprises here, by way of example, a drive 34 for opening and closing the mold and a drive 35 for raising or lowering a container comprising fibers. The filling and closing device 32 comprises here, by way of example, a drive 36 for a movement of filling nozzles and a drive 37 for a movement of closing heads.

1. Method for sterilizing raw material for producing at least a part of a container comprising fibers, wherein the method comprises:
providing the raw material,
sterilizing the raw material to form sterilized raw material.

2. Method according to claim 1, wherein the sterilization comprises a physical and/or electrothermal sterilization, wherein the physical sterilization takes place by means of a physical sterilizer.

3. Method according to claim 2, wherein the sterilization comprises thermal heating, wherein the thermal heating takes place by means of a flash pasteurization device.

4. Method according to claim 2, wherein the sterilization comprises chemical sterilization, wherein, chemical sterilization takes place by means of addition of chemical disinfectant, wherein the chemical disinfectant comprises H₂O₂, O₂, O₃, NaClO₂, NaClO, HClO, HOCl-water or electrochemically activated water.

5. Method according to claim 1, wherein the raw material comprises fibers.

6. Method according to claim 1, wherein the raw material comprises fibers and a fluid, wherein the fibers comprise natural fibers, wherein the fluid comprises water, wherein the fluid comprises additives.

7. Sterilized raw material for producing at least a part of a container comprising fibers, wherein the sterilized raw material was obtained by means of the method according to claim 1.

8. Method for producing a container comprising fibers using the sterilized raw material according to claim 7.

9. Container comprising fibers, wherein at least a part of the container comprising fibers comprises sterilized raw material which was obtained by means of the method according to claim 1.

10. Container comprising fibers according to claim 8, wherein the container comprising fibers was produced by means of the method according to claim 8.

11. Device for sterilizing raw material for producing at least a part of a container comprising fibers, wherein the device is designed to carry out the method according to claim 1.

12. Device according to claim 11, further comprising a feed device for raw material and a sterilizing device for sterilizing the raw material.

13. Device according to claim 12, wherein the sterilizing device comprises at least one of the following:
a microwave radiation generation device,
an ohmic heater,
an induction heating device, —an ultraviolet radiation generation device,
an infrared light generation device,
a high frequency generation device,
a pulsed electric field generation device,
a plasma generation device,
a high pressure generation device,
a flash pasteurization device,
a feed device for feeding chemical disinfectant.

14. Container treatment plant comprising a provision device for providing a sterile pulp comprising fibers, a production device for producing a container comprising the fibers from the sterile pulp comprising fibers, and a filling and closing device for filling the sterile container comprising fibers with product and for closing the filled sterile container comprising fibers, and at least one aseptic room which is designed to maintain a defined aseptic environmental condition for the sterile container comprising fibers, and which extends from the provision device to the filling and closing device.

15. Container treatment plant according to claim 14, wherein the at least one aseptic room is a room having a low-contamination environment,
wherein the low-contamination environment is a clean room,
wherein the low-contamination environment is obtained by one or combinations of the following measures:
the low-contamination environment is subjected to an overpressure compared to the ambient atmosphere, by providing fine filters for blowing in air filtered by the fine filters into the aseptic room at least during an operating mode of the container treatment plant,
arranging drives for a very wide variety of movements of a mold for producing the container comprising fibers and/or transport elements outside the aseptic room,
cleaning and/or sterilizing a surface of an inner wall of the aseptic room at regular intervals, cleaning and/or sterilizing inner and outer sides of the mold and/or the filling nozzles and/or of closing heads, cleaning and/or sterilizing surfaces of internal walls of the machine protection,
sealing the low-contamination environment against the ambient atmosphere, by providing a water lock or a rubber seal as a seal in a carousel-type machine for sealing the rotating part relative to the stationary part of the carousel-type machine.

16. Method according to claim 1, wherein sterilization by means of the physical sterilizer includes one or more of by means of microwave radiation, by means of an ohmic heater, by means of ultraviolet radiation, by means of high-frequency heating, by means of a pulsed electric field, by means of plasma and/or by means of high pressure.

17. Method according to claim 1 wherein the fibers include dry fibers, wherein the fibers comprise natural fibers.
